(19) United States
(12) Patent Application Publication
(10) Pub. No.: US 2019/0030003 A1
(43) Pub. Date: Jan. 31, 2019

(54) GASTROINTESTINAL-PROTECTIVE FORMULATIONS FOR ORAL DELIVERY OF PROTEINS AND PEPTIDES

(71) Applicant: BIODURO, LLC, San Diego, CA (US)

(72) Inventors: James Blair WEST, San Diego, CA (US); Ke SHI, San Diego, CA (US)

(73) Assignee: BIODURO, LLC, SAN DIEGO, CA (US)

(21) Appl. No.: 15/927,977

(22) Filed: Mar. 21, 2018

Related U.S. Application Data
(60) Provisional application No. 62/474,311, filed on Mar. 21, 2017.

(51) Int. Cl. A61K 31/4178; A61K 9/48; A61K 9/16; A61K 9/00

(52) U.S. Cl. CPC A61K 31/4178; A61K 9/0053; A61K 9/1652; A61K 9/48

(57) ABSTRACT

Disclosed herein are processes for formulating a protein or a peptide for oral delivery, the process comprising the steps of: obtaining a powder containing the protein or peptide; obtaining an amount of an enteric polymer, wherein the enteric polymer is neutralized so that it is soluble in water; optionally obtaining at least one excipient; mixing the protein or peptide, the neutralized enteric polymer, and optionally the at least one excipient to obtain a First Dispersion; granulating the First Dispersion on a fluidized bed to obtain a Second Dispersion; whereby the protein or peptide in the Second Dispersion undergoes less than 50% proteolysis under gastric conditions.

### CROSS-REFERENCE TO RELATED APPLICATIONS

This application claims priority to U.S. Provisional Application Ser. No. 62/474,311, filed on Mar. 21, 2017, by James Blair West et al., and entitled “GASTROINTESTINAL-PROTECTIVE FORMULATIONS FOR ORAL DELIVERY OF PROTEINS AND PEPTIDES,” the entire disclosure of which is incorporated herein by reference, including all the drawings.

### FIELD OF THE INVENTION

The present invention is in the field of generating oral formulations for peptides and proteins, and other active pharmaceutical ingredients (APIs) that are sensitive to hydrolysis or denaturation under acidic conditions and inactivation by gastric enzymes such as pepsin.

### BACKGROUND OF THE DISCLOSURE

Increasingly, proteins and peptides are being developed for therapeutic purposes, and for approaches that permit, or require, oral delivery. The therapeutic target for certain active pharmaceutical ingredients (APIs) of interest exert their therapeutic effect in the GI tract. Therefore, the historically recognized poor absorption of these molecules is not an issue. However, there are significant physiological barriers for this class of APIs reaching the site of action in the GI tract intact. Chief among these is the proteolytic activity present in the stomach provided by the proteases such as pepsin. Consequently, orally administered protein and peptide APIs are rapidly degraded when exposed to the proteases in the gastric juice.

Approaches have been developed that prevent the APIs from dissolving in the gastric juice and delay their release until the dosage form has left the stomach and entered the intestine where the rising pH inactivates the pepsin. One approach is to coat the dosage form (tablet, capsule, beads) with an enteric polymer, i.e., a polymer designed to not dissolve in the acidic conditions of the stomach (pH 1.3), but eventually dissolve when the dosage form leaves the stomach and encounters the higher pH of the small intestine (pH 5 in the duodenum; pH 6-7 further into the small intestine). Using this approach, the API is not released in an environment that will lead to its rapid degradation. Accordingly, coated beads, tablets or capsules are generated that do not dissolve or dissolve slowly in the stomach, but do dissolve in the small intestine. (Fieker, et al., 2011.)

Another approach currently used in the art is to microencapsulate the protein or peptide. This process uses complex emulsions (e.g., water:oil:water emulsions) with the API constrained in the inner water phase, and the oil phase containing the polymer that will make up the encapsulation material. The final form of this process is small spherical microcapsules containing the API. These microcapsules can be collected, optionally dried, and formulated in a variety of ways for oral delivery. (Meng, et a., 2003.)

Problems with the current, aforementioned approaches include multistep processes that can lower the levels of activity of the API (through damage to the protein or peptide structure by partial or complete denaturation or by non-specific aggregation). Also the achievable dose of the API can be limited by the structures generated to provide the protection (such as the mass of bead cores and subsequent protective layers, or the low ratio of encapsulated volume to total formulation volume achieved in microencapsulation.

### SUMMARY OF THE INVENTION

Disclosed herein are processes for formulating a protein or a peptide for oral delivery, the process comprising the steps of: obtaining a powder containing the protein or peptide; obtaining an amount of an enteric polymer, wherein the enteric polymer is neutralized so that it is soluble in water; optionally obtaining at least one excipient; mixing the protein or peptide, the neutralized enteric polymer, and optionally the at least one excipient to obtain a First Dispersion; granulating the First Dispersion in a fluidized bed to obtain a Second Dispersion; whereby the protein or peptide in the Second Dispersion undergoes less than 50% proteolysis under gastric conditions.

### BRIEF DESCRIPTION OF THE DRAWINGS

FIG. 1 is a graph showing the activity release under simulated GI tract conditions. Simulated gastric phase (first 30 minutes), contains 1 mg/mL of pepsin.

FIG. 2 is a graph showing the enteric protection and activity release by neutralized HPMCAS formulation containing 18% and 30% beta-lactamase (1.1×10⁷ units/L pepsin in gastric dissolution medium).

FIG. 3 is a graph showing the enteric and activity release by neutralized HPMCAS formulation containing 30%, 40% and 50% beta-lactamase (1.1×10⁷ units/L pepsin in gastric dissolution medium).

FIG. 4 is a graph showing the effects of different matrix polymer grades and API loading on activity release. Simulated gastric phase (first 30 minutes) contains 1 mg/mL pepsin.

FIG. 5 is a graph showing the effects of adding citric acid to the capsule blend on activity release. Simulated gastric phase (first 30 minutes) contains 1 mg/mL pepsin.

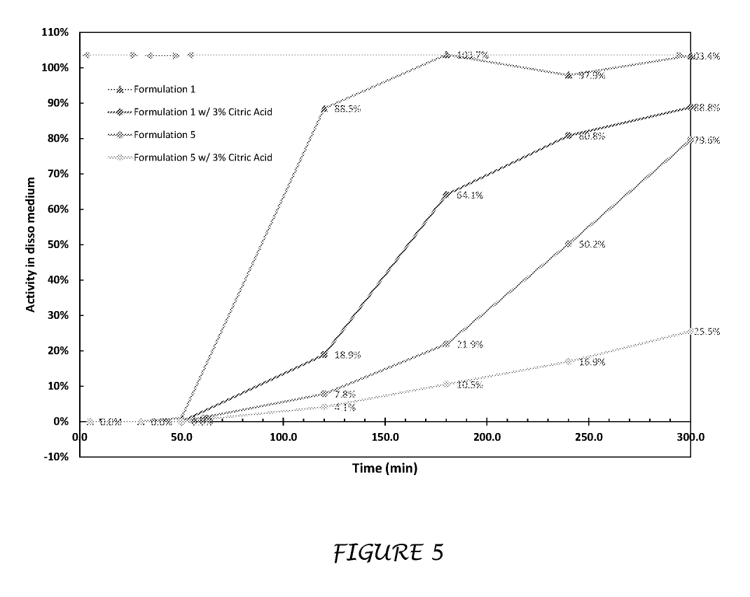

FIG. 6 is a graph showing the results of the multi-stage dissolution test to spray dried chymotrypsin capsule Formulation 6 (no pepsin in dissolution medium).

### DETAILED DESCRIPTION OF THE EMBODIMENTS

Disclosed herein is a description of materials and processes used in the manufacture of novel oral dosage forms that have the following beneficial properties:
1) Partial or complete protection of the API from some or all enzymes in the GI tract whose interaction with the API lowers the amount of the API at the desired site of action.
2) Minimizing the process steps required to prepare the finalized dosage form from the API feed stock (either liquid or solid) and utilizing processes amenable to maintaining protein structure and activity.
3) Maximizing the recovered activity of the API, especially for those API' s whose activity depends on the retention of secondary and tertiary structure in solution.
4) Generating a powder that is directly incorporated into a final dosage form by using a single spray-drying step on an aqueous solution comprising the dissolved API and other excipients. The resultant spray-dried powder provides protection of the API from enzymatic degradation after dosing, and optionally provides different rates of dissolution of the API, thus providing different API release profiles. The nature and amounts of the excipients are adjusted to give the different release profiles.

### Definitions:

Gastrointestinal-protective formulation: an oral formulation containing an enzyme sensitive API (such as a peptide or protein), which formulation provides complete or partial protection from naturally occurring proteolytic or other enzymatic activity on the API in the GI tract, thereby increasing the amount of active API surviving the gastric and intestinal environments to reach the site of action.

Proteolysis Sensitive API: an (API) containing amide or ester moieties that are catalytically hydrolyzed by naturally occurring gastrointestinal enzymes, the resultant hydrolysis of which leads to a partial or complete inactivation of the biological activity of the API.

Enteric Polymer: a polymer designed to be a film-forming coating that does not dissolve in solutions at typical gastric pH. Typically, enteric polymers are multifunctional polymers containing mixtures of hydrophobic side chains and pendant carboxylic acids, the ratio of which gives a polymer that is water insoluble when the carboxylic acids are mostly protonated (below pH 3), but is water soluble when most of the carboxylic acids are deprotonated and, thus, negatively charged (above ˜pH 5 or so).

Fluidized Bed: a collection of powder suspended in a device by a column of moving air, used for drying and granulation processes in pharmaceutical processing.

Fluidized Bed Wet Granulation: the binding of smaller powder particles into larger particles (or granules) by spraying an aerosol of a binder onto a fluids bed of the particles.

First Dispersion: a spray-dried dispersion of neutralized enteric polymer, the protein API and optional additives to improve stability or modify eventual drug release.

Second Dispersion: agglomerated particles of the First Dispersion, with improved flow, release and manufacturability characteristics.

About: throughout the present disclosure the term “about” a certain value means that a range of value ±10%, and preferably a range of value ±5%, is contemplated. Thus, for example, having about 70% w/w of a component includes the component being present between 63% and 87%, and preferably between 66.5% and 73.5%. Furthermore, when about a range is given, it is understood that the word “about” qualifies both termini of the range. Thus, for example “about 7-10” means “about 7 to about 10.”

Disclosed herein are processes that provide a novel method for generating a formulation for a protease-sensitive API (such as a protein or peptide) that will protect the API from proteolysis prior to the API reaching the desired site of activity in the GI tract. Among the characteristics of the novel formulation is that it can provide high amounts or protected API and provide higher levels of residual activity of sensitive biomolecules after processing.

In some embodiments, the water soluble poly-salt of commercially available enteric polymers used for drug products requiring protection from gastric conditions is formed by neutralizing a suspension of the polymer in water using a dilute solution of a base, such as sodium hydroxide, or other water soluble bases. The polymers are uniformly carboxylic acid containing polymers designed to be soluble in water if a majority of the carboxylic acids are deprotonated, and conversely, insoluble in water if a majority of the carboxylic acids are deprotonated.

In some embodiments, such enteric polymers are used, but not as a protective coating, but as a gelling matrix with protects proteolysis-sensitive API's. In one embodiment, an enteric polymer is suspended in water and slowly deprotonated with a base such as dilute NaOH, until the polymer dissolves, essentially forming a solution of the sodium salt of the polymer.

In some embodiments, the above solution of the polymer is isolated as a solid by spray drying it to remove the water. The resultant solid powder is the poly-valent salt of the enteric polymer. In some embodiments, this material is soluble in pH neutral water, but immediately forms a viscous gel upon encountering acidic pH (typically pH 3-4 or below).

Thus, in some embodiments, the core of the formulation is obtained by the deprotonation of a commercially available enteric polymer, followed by the isolation of the polymer solid by spray drying to generate the acid-gelling polymeric material.

Adding a dilute base to an aqueous suspension of the polymers deprotonates increasing numbers of the carboxylic acids attached to the polymer backbone and the polymer becomes soluble in the aqueous phase. The protein API is dissolved in water with a certain amount of deprotonated enteric polymer, then spray-dried to produce a First Dispersion. The First Dispersion is then processed to increase particle size. Additional API-protective components are included through wet granulating of the First Dispersion by spraying a solution of a binding polymer onto a fluidized bed of the First Dispersion. The resulting material exhibits high protein activity recovery (low protein degradation), enhanced protection to gastric conditions, and is manufacturable in a variety of dosage forms.

The inventors have surprisingly found that the First Dispersion is not sufficient for generating gastro-protective formulations of gastric sensitive protein APIs due to at least two technical issues. First it was found the powder produced in the First Dispersion provides protection only if loosely packed into a standard capsule, and if not exposed to model gastric fluid in this manner. For example, when added to model gastric fluid as a free powder, the First Dispersion afforded only partial or low protection from acidic conditions or from digestion by gastric enzymes, such as pepsin. It was observed that more firmly packing the First Dispersion into capsules led to incomplete release of the protein API, as measured by the recovered activity of the enzymes treated in this way.

In addition, it was determined that, in some embodiments, when the First Dispersion was used untreated, it was not amenable to standard dosage form manufacturing processes. The powders possessed very low specific density, and exhibited very poor powder flow characteristics. It was surprisingly found that the use of the standard approaches known in the art for addressing poor flow or low density, such as dry granulation by roller compaction (Leane, et al. 2012), produced low recovery of the protein API as judged by recovery of activity of enzymes.

We have discovered, however, that in some embodiments the First Dispersion can be treated by a process of wet granulation. Spraying a solution of a binder protein on a fluidized bed of the First Dispersion produces larger particles with good powder flow. The larger particles lend themselves to pharmaceutical manufacturing processes, and release near total activity of the protein API upon dissolving. In some embodiments, the binder polymer used in the granulation process is itself an enteric polymer, providing additional protection. In some embodiments, the enteric binding polymer is sprayed in an organic solution (as the protonated enteric polymers are not soluble in water unless neutralized), while in other embodiments, the enteric binding polymer is dissolved in a neutralized form from an aqueous solution. In certain embodiments, the choice of which embodiment is used depends on the protein API being used and the desired release profile.

Examples of enteric polymers used to prepare the First Dispersion or the Second Dispersion/Granulation include, but are not limited to, hypromellose acetate succinate (HPMCAS, Shin Etsu, Tokyo, Japan); poly(methacrylic acid-co-ethyl acetate) (Eudragit L30, Eudragit L100, Evonik, Essen, Germany); poly(methacrylic acid-co-methyl methacrylate) 1:1, (Eudragit L12.5, Evonik); poly(methacrylic acid-co-methyl methacrylate) 1:2 (Eudragit S100, Evonik), among others. Each of these polymers is insoluble in water at low pH (1-3) but becomes soluble at pH 5 or above.

In one embodiment, HPMCAS MF (hydroxypropyl-cellulose acetate succinate, MF grade, hypromellose acetate succinate. Shin-Etsu, Tokyo, Japan) is the enteric polymer used for the First Dispersion. The enteric polymer is suspended in water, and a dilute solution of base (NaOH) is added with stirring until the polymer dissolves. This solution is spray-dried, to remove the water, giving a powder that is essentially the multivalent sodium salt of HPMCAS (HPMCAS⁻Na⁺). The powder is then dissolved in water, along with the API, and optionally other components. Optionally, the solution may also contain buffer salts (e.g. NaHPO₄) (to control pH during processing or upon dissolution), protein structure stabilizers (e.g. trehalose, Tween 80) (to maintain the structure and activity of the protein), additives that can control the dissolution rate of the resultant gel at higher pH, or other functional additives. The solution is spray-dried to form a dry powder to give the First Dispersion

The First Dispersion powder is then wet granulated, where the powder is suspended in circulating air and sprayed with a solution of a binder polymer. This serves to bind the powder particles into larger granules. Such granules can provide enhanced protection of the API from gastric conditions (by coating the powder particles with a layer of binder polymer), produce larger particles with better powder flow, and facilitating the further manufacture of a final dosage form using standard pharmaceutical manufacturing techniques. Additionally, the binder coating can serve as an additional control mechanism on the release profile of the API into solution by modifying the dissolution of the granules when the desired site of the GI tract is reached. In some embodiments, an enteric polymer (e.g., HPMCAS in an organic solvent) is used as the binder polymer. In another embodiment, an aqueous solution of an enteric polymer salt is used, which may be advantageous in cases where the use of an organic solvent in processing has a negative effect on protein activity.

In some embodiments the resultant granulated powder is encapsulated in standard gelatin, capsules, formulated as a powder for suspension, or even, with some protein APIs, be formulated in a tablet.

Upon administration, the formed granules begin to interact with the acidic gastric environment. In some embodiments, the enteric polymer salt exposed to the acidic environment begins to dissolve, but upon interaction with the low pH, will phase separate, forming a viscous gel. In some embodiments, the interaction of the enteric polymer salt is also affected by the nature and amount of the binding polymer used for the granulation process. For instance, using a protonated enteric polymer delays or slows the interaction of the enteric polymer salt with the acid in the gastric environment. Upon exiting the stomach, the pH of the GI tract rises, and the enteric polymer in the granules, both the matrix of the First Dispersion, and the binding polymer forming the granule, dissolve, thereby freeing the API. In some embodiments, the rate of this dissolution is controlled and adjusted by, for example, the selection of the enteric polymer used in the First Dispersion, additives that slow the dissolution of the First Dispersion even under pH conditions in this the polymer would dissolve readily, and/or the nature and the amount of the binding polymer used to generate the granulated Second Dispersion.

### EXAMPLES

### Example 1

### First Dispersion

To test the expected performance of the First Dispersion alone, a dissolution/activity release assay was established to mimic the changing gastrointestinal environment the dosage form would encounter upon administration. A USP Type II dissolution apparatus is used, and the initial dissolution media is USP defined ‘simulated gastric fluid (SGF), with enzyme. This consists of 0.1 g to 1 g of porcine pepsin (2000-3500 units/mg specific activity) 7 mL HCl per liter of water (final pH 1.2). Dosage forms (powders, capsules) are added to the dissolution bath (paddle speed 75 rpm), and aliquots removed at various times and the activity of the enzyme determined. After 30 minutes, the pH is raised to 5.0 with NaOH for 10 minutes. Finally, the pH is adjusted to 7.0. These pH changes are meant to simulate gastric environment, then passing out into the duodenum, and finally to represent the middle small intestine. An alternate method starts the gastric conditions at pH 3 with pepsin, to elucidate the possible variation of gastric pH and its effect on formulation performance. Under these pH conditions, the pepsin is expected to be fully active at pH 1 and 3, about 40% active at pH 5 and totally inactive at pH7.

HPMCAS-MF (Shin-Etsu) was suspended in water (5 wt %). With stirring, 0.1M NaOH was slowly added, care being taken that the pH did not rise above about pH 8 in the solution. After complete dissolution of the polymer a slightly hazy clear solution was obtained. To this solution was added beta-lactamase (Fujifilm Diosynth, Morrisville, N.C.) (in a buffered, liquid preparation) to a concentration of about 20 mg/mL. This solution was spray-dried on an Anhydro MS-35 spray-drier. The overall recovery of material was 85% based on solids content of the spray solution.

Spray drying was conducted on Anhydro® MS-35 with a two-fluid nozzle (1.2 mm nozzle size and 6 mm gap). The atomization air pressure was about 50 psig and process nitrogen gas flow was about 30-35 kg/hr. Inlet temperature varied between about 140-170° C. in order to maintain an outlet temperature at about 55-70° C. In a typical development batch, about 300-800 gram of solution containing about 6% solid was spray dried, yielding about 15-30 gram of SDD sample within about 10-30 min. A representative solution composition and spray drying parameters are included in Table 1.

TABLE 1
A representative spray drying solution composition and parameters

| | | |
|---|---|---|
| Spray drying solution | DI water | 74.1% |
| composition to produce SDD | HPMCAS MF | 4.9% |
| with 30% w/w drug | beta-lactamase solution (100 | 21.0% |
| loading (% w/w) | mg/mL) | |
| Spray drying parameters | Inlet temperature (° C.) | 170 |
| | Outlet temperature (° C.) | 71 |
| | Atomization air pressure | 49 |
| | (psig) | |
| | Process air flow rate (kg/hr) | 35 |
| | Solution feed rate (g/min) | 20 |

The resultant powder was filled into either HPMC capsules (Capsugel, Greenwood, S.C.). The flow characteristics were determined by measuring Carr's Index and Angle of Repose as defined in U.S. Pharacopeial Convention Title 1174 (USP <1174>), and determined to be very poor (Can's Index of 35).

Released enzyme activity was measured by measuring the hydrolysis of a chromophoric beta-lactamase substrate CENTA (Jones, et. al., 1982). Aliquots from the dissolution are mixed with a reaction buffer and the rate of reaction determined spectrophotometrically.

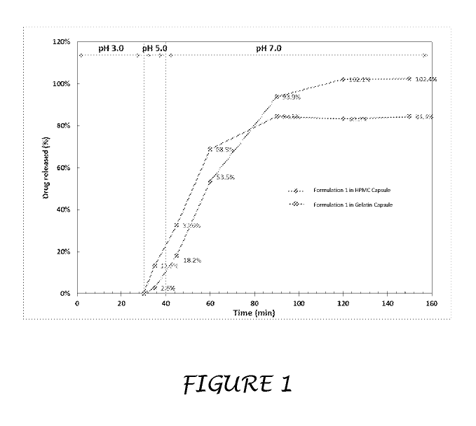

FIG. 1 presents the release of beta-lactamase activity from the capsule. Under these conditions, the gelatin capsule dissolved somewhat faster than the HPMC capsule, releasing some of the active while the pepsin was still active. The total activity released from the formulation is lower, due to slightly higher levels of degradation of the API. However, both formulations show good protection of activity (indicated by total activity recovered), and show good rates of release. The First Dispersion powder alone (not packed in to an HPMC capsule) when added to a pH 1.2/pepsin solution which was then adjusted to pH 6.8, showed no residual activity.

### Example 2

### First Dispersion, Enzyme Loading

We demonstrated that high amounts of beta-lactamase as the API can be included in the First Dispersion, with retention of recoverable activity even up to 50% API loading (Table 2).

TABLE 2
Composition of HPMCAS matrix formulation

| | | Formu- | Formu- | Formu- | Formu- |
|---|---|---|---|---|---|
| Lot# | | lation 1 | lation 2 | lation 7 | lation 8 |
| Percentage in | SYN-004 | 18.0% | 30.1% | 40.1% | 50.0% |
| formulation | HPMCAS M/F | 80.4% | 68.4% | 59.9% | 50.0% |
| (w/w %) | Phosphate buffer | 1.6% | 1.5% | — | — |
| | salts (optional) | | | | |

| | | | | |
|---|---|---|---|---|
| Measured Enzyme | 17.2% | 28.9% | 40.5% | 48.3% |
| Concentration in Formulation | | | | |
| (Determined by Activity in | | | | |
| CENTA Assay) | | | | |
| Activity recovery | 96% | 96% | 101% | 97% |
| Activity after Pepsin exposure | 97% | 98% | 77% | 75% |

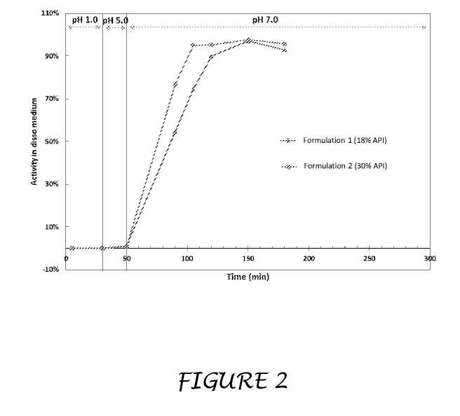

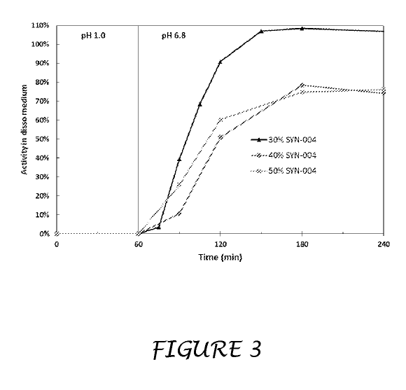

However, it was observed that at the highest drug loadings (40% and 50%), protection from pepsin exposure was not as complete, with residual activity levels falling to around 75%, as shown in FIG. 2 and FIG. 3.

### Example 3

### Granulated Dispersions (Second Dispersion)

The powder flow property of neutralized HPMCAS formulation was characterized. Beta-lactamase SDD was very fine and fluffy powder with bulk density of about 0.15-0.20 g/cm³, tapped density of about 0.33-0.35 g/cm³. The calculated Can's index ranged between 30 and 35, indicating the powder has very poor flow characteristics, indicating that this material would be very difficult to process using standard pharmaceutical manufacturing processes. Can's index is calculated using the following formula:

Carr's index=100×(1−bulk density/tapped density)

To address this, dry granulation by slugging and roller compaction was evaluated. However, although the flow properties of powder were improved, the dry granulation process reduced the activity by about 15%-20% using force between about 500-1700 psi during the processing. This suggested that the enzyme could be sensitive to mechanical stress. Wet granulation by fluid bed, on the other hand, seemed to be more suitable for beta-lactamase SDD.

The granule obtained from a typical fluid bed granulation provided material that had a calculated Carr's index of about 20-24, indicating good flow.

To generate this material, a dispersion of Beta-lactamase in neutralized HPMCAS was blended with about 1% w/w fumed silicon dioxide (Airosil 200) and loaded onto a Mini-Glatt fluidized bed with a top-spray configuration. Coating solution was prepared by dissolving HPMCAS in acetone to about 4% w/w. During granule coating, the fluidizing air flow rate was preheated to about 55-60° C. before entering powder bed. Coating solution was sprayed downwards onto the powder bed through a two-fluid Schlick nozzle fed by a peristaltic pump from a reservoir positioned on a balance. The liquid flow rate is controlled by the pump revolution setting and by the continuous recording of reservoir weight. The air flow rates, temperatures and pressure were monitored by the internal sensors of the Mini-Glatt. Product temperature, monitored by thermometer, was kept between about 35-37° C. through the process. The typical batch size was about 10 g.

The enzymatic activity was measured using the CENTA assay method described before. The enteric protection and activity release was evaluated using the dissolution method described. Specifically, beta-lactamase granules were directly added to two-stage dissolution bath and mixed with a Type II USP dissolution apparatus at a rotation speed of about 100 rpm at about 37.0° C. The dissolution medium for acid stage consisted of about 750 mL of 0.1N hydrochloric acid solution. Optionally, 2 g/L sodium chloride and 7.7×10⁴ units/L pepsin were added to the acid dissolution medium to form a simulated gastric environment. Dissolution at the acid stage lasted for 120 min, followed by neutralizing the medium with 250 mL phosphate buffer concentrate to pH 6.8. Buffer stage dissolution continued for another 4 hours. Aliquots of the resulting solution were withdrawn at the time-points shown in the results below, centrifuged, diluted and assayed using CENTA assay method. The result was expressed as percentage of dissolved activity from the total activity of the samples in the capsule.

Several prototypes of enteric-coated granules were produced from different First Dispersions of spray beta-lactamase and coated with either HPMCAS-M or HPMCAS-H (Table 3). During the coating process for each lot, samples were collected during the process and assigned with sub-lot number “A” through “C”. For example, Formulation 9A was collected at earlier stage during coating than Formulation 9B, thus had less coating layer and higher content of enzyme and higher activity.

TABLE 3
Composition of enteric-coated beta-lactamase granules

| | Prototype 1 | Prototype 2 |
|---|---|---|
| Composition of Spray | 30% w/w beta- | 50% w/w beta- |
| Dried beta-lactamase | lactamase in | lactamase in |
| | neutralized | neutralized |
| | HPMCAS-M | HPMCAS-H |
| Coating polymer | HPMCAS-H | HPMCAS-H |
| Residual Activity | 105% | 90% |
| Two Stage Dissolution | | |
| Stage 1: 2 hours pH 1 + | | |
| pepsin (7.7 × 10⁴ units/L) | | |
| Stage 2 pH 6.8 | | |
| Carr's Index for | 22 | 21 |
| Granulated Dispersion | | |

These results provide the basis for some of the formulations disclosed herein, a First Dispersion containing the active, and optionally release-controlling excipients, and the Second dispersion containing the First, providing good processibility and additional protection from gastric conditions, and control of release of the API.

### Example 4

### First Dispersion Modified Release of Activity

In some embodiments, modifying the rate of release maybe desirable. Two methods for slowing the release are described herein. The method of preparation of the formulation is the same, but using a different grade of the enteric polymer, one that has a different ratio of hydrophobic groups to carboxylic acids on the polymer. Also, rates can be altered by adding excipients that are acids or bases into the formulation.

FIG. 2 presents the activity release from formulations where the matrix polymer is HPMCAS-MF as in Example 1, and in HPMCAS-HF, a grade of HPMCAS that dissolves at higher pH than the MF grade. Also examined is the effect of different API loadings in the formulations, 18% or 23% (Formulation 2, 3, 4 and 5).

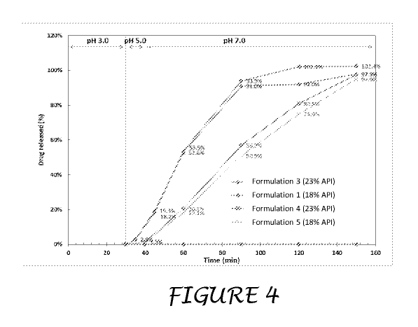

The results presented in FIG. 4, where using the HPMCAS-HF polymer slows the release of activity over that of the formulation using HPMCAS-MF. This can be explained by the fact that the HF grade is designed to dissolve at a higher pH than the MF grade, so the release of activity is slowed in relation to the formulation using the MF grade.

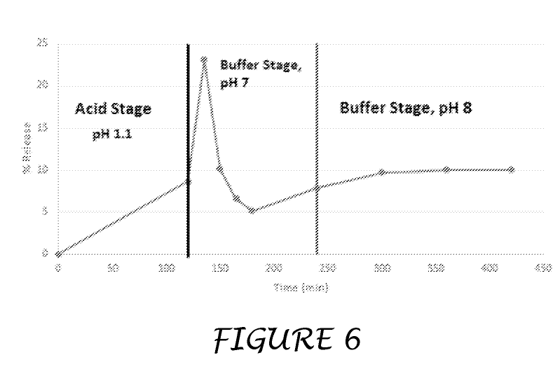

Beyond changing the grade or nature of the polymer, another level of control over activity release rates is to include additives that effect the rate at which the dosage form dissolves even when conditions of a neutral pH are reached in the GI tract. In one embodiment of the invention, solid acids can be added to the First Dispersion to maintain a lower local pH within the gel as compared to the surrounding environment to slow dissolution of the gel and release of the API. In the example presented in FIG. 6, 3% (wt:wt) of citric acid added to the First Dispersion provided protection from pepsin, but slowed the released of enzyme activity into solution at pH 7.

### Example 5

### Other Proteins. First Dispersion, Chymotrypsin

Chymotrypsin is a serine endopeptidase whose activity can be quantified using colorimetric reaction method. For this project, chymotrypsin served as a low-cost alternative to IAP to establish the spray drying parameters. The formulation and production parameters for spray dried chymotrypsin was summarized in Table 4. A good activity recovery at about 91.5% was obtained suggesting that the used production parameters were appropriate to preserve chymotrypsin activity.

The enzymatic assay for chymotrypsin was as given. Working standards and sample solutions, both in the range of 10 to 160 μg/mL, were prepared in 0.001 N hydrochloric acid solution. The substrate for chymotrypsin, N-succinyl-L-phenylalanine-p-nitroanilide (SPNA, Sigma-Aldrich, St. Louis, Mo.) was dissolved in buffer (50 mM Tris, pH 7.5) to 1 mM. To each well of a 96-well plate, 50 μL of working standard or sample solution was transferred, and then 200 μL SPNA solutions were simultaneously added. After 10-min reaction at 25° C., the reaction was stopped by adding 50 μL 30% acetic acid. The absorbance at 410 nm for each well was measured. A linear standard curve was obtained by plotting the absorbance against the standard concentration. The activity of samples was calculated using the standard curve and expressed as weight percentage of active enzyme in samples

TABLE 4
Composition and enzymatic activity of spray dried chymotrypsin

| | | Formu- |
|---|---|---|
| Lot# | | lation 6 |
| Percentage in | chymotrypsin | 30.3% |
| formulation (w/w %) | HPMCAS HF | 69.7% |
| Spray drying | Solid content in spray drying solution | 6.6% |
| parameters | Inlet temperature | 170° C. |
| | Outlet temperature | 53° C. |

| | |
|---|---|
| Activity recovery | 91.5% |

The spray dried chymotrypsin powder was manually filled into size 1 HPMC capsule to reach a dose of 60 mg enzyme per capsule. The capsule was analyzed in a multi-stage dissolution, where medium was pH 1.0 for two hours, then pH 7.0 for two hours and pH 8.0 for two hours. The final adjustment to pH 8 was to ensure complete dissolution of HPMCAS-H matrix. The percentage of detected activity in the medium to the total activity in the capsule over time was plotted in FIG. 4. By the end of 2-hr acid stage, about 8% activity was leaked into dissolution medium. Within 15 min following pH neutralized to 7.0, the activity in the medium rose to about 23%, then dramatically decreased to 5% by one hour after pH neutralization, then slowly rose back and stayed at about 10% towards the end of the test (FIG. 6). Chymotrypsin is well known for its capability undergoing autolysis that degrades and inactivates the enzyme itself. Depending on the species and assay method, different pH dependency has been report for its autolysis. Generally, enzymatic activity of chymotrypsin is low (<10%) at pH about 5.5 or lower, and pH about 10 or higher. The optimum pH for its activity is between about 7-8. The competition between dissolution/release and autolysis of chymotrypsin explained fluctuated activity in dissolution medium after pH neutralization. Right after pH neutralization, the HPMCAS-H matrix began to dissolve and release the activity into medium. In about 15 min, the autolysis took over and deactivate the dissolved enzyme in the medium. As the level of remained active enzyme in the medium decreased, the autolysis reaction slowly diminished eventually left 10% activity in the medium.

### Example 6

### Other Proteins. First Dispersion, Alcohol Dehydrogenase (ADH)

Alcohol dehydrogenase (ADH) facilitates the interconversion between alcohols and aldehydes or ketones with the reduction of nicotinamide adenine dinucleotide (NAD⁺ to NADH and the activity can be quantified using colorimetric reaction method as chymotrypsin. ADH was chosen as a model enzyme because it is readily available and requires zinc ion cofactors for full activity. ADH was examined to see if the zinc ion cofactors could be incorporated into the Dispersion formulation. Slightly different levels of zinc content in the native structure of ADH were suggested by different studies and one suggested level was 2.2 mg of zinc ions per gram of ADH protein. In this study, four prototypes of spray dried ADH were produced to compare the effects of excipients on the activity recovery (Table 5). The activity recovery was calculated using Equation 1.

$$Activity recovery = \frac{\begin{matrix}Activity of spray dried \\ product measured by Assay kit\end{matrix}}{\begin{matrix}Activity of pure enzyme measured by Assay kit \times \\ weight percentage of enzyme loading\end{matrix}} \times 100 \% . \qquad Equation 1$$

For the first prototype Formulation 10, same formulation and production parameters for spray dried chymotrypsin was adapted, except that the enzyme loading was reduced to 5.0%. However, only 52% activity was recovered. Considering that deficiency of cofactor and relatively higher processing temperatures might have damaged the activity, in the following prototype Formulation 11, zinc was added and processing temperatures were significantly lowered, resulting in only slight increase of activity recovery to 66%. The ADH enzyme shall naturally contains zinc ions and additional zinc ions in Formulation 11 seemed to not effectively increase the activity recovery, suggesting that heating may dominate the damage to the enzymatic activity. Given that further lowering the spray drying temperatures increased the risk of high water content in the product, commonly used protein stabilizers, i.e. polysorbate 20 and trehalose, were co-sprayed with the enzyme, polymer, buffer salts and zinc ions in Formulation 12 and Formulation 13. Addition of the stabilizers showed obvious improvement to the activity recovery. Among which, Formulation 13 with addition of trehalose resulted in an excellent activity recovery at 125%. The value was over 100%, possibly due to variations caused by the non-optimized and non-validated ADH enzymatic assay. A commercial ADH assay kit (Sigma-Aldrich, St. Louis, Mo.) following NADH production was utilized for all enzyme assays.

TABLE 5
Composition and enzymatic activity of
spray dried alcohol dehydrogenase.

| | | Formu- | Formu- | Formu- | Formu- |
|---|---|---|---|---|---|
| | | lation | lation | lation | lation |
| Lot# | | 10 | 11 | 12 | 13 |
| Percentage in | ADH | 5.0% | 5.0% | 5.0% | 5.0% |
| formulation | HPMCAS HF | 95.0% | 92.0% | 90.4% | 90.4% |
| (w/w %) | Zinc sulfate | — | 1.0% | 0.6% | 0.6% |
| | heptahydrate | | | | |
| | Monosodium | — | 2.0% | 2.0% | 2.0% |
| | phosphate | | | | |
| | monohydrate | | | | |
| | Polysorbate 20 | — | — | 2.0% | — |
| | Trehalose | — | — | — | 2.0% |
| Spray | Inlet | 170° C. | 145° C. | 145° C. | 145° C. |
| drying | temperature | | | | |
| parameters | Outlet | 51° C. | 45° C. | 47° C. | 47° C. |
| | temperature | | | | |

| | | | | |
|---|---|---|---|---|
| Activity recovery | 52% | 66% | 80% | 125% |

These results demonstrate that the presence of zinc ions is important for full recovery of activity in the dispersion system.

### Example 7

### Other Proteins. First Dispersion: Calf Intestinal Alkaline Phosphatase (cIAP)

CIAP requires both zinc and magnesium for activity. The equivalent to 0.1 mM of zinc ions and 1.0 mM of magnesium ions were added to the spray drying solution of cIAP (5% total solid content) to ensure more than sufficient ions present to maintain the native conformation of cIAP. The formulation in spray dried cIAP and activity recovery is summarized in Table 6. Activity of the enzyme was determined with a commercial assay kit, SensoLyte® pNPP Alkaline Phosphatase Assay Kit *Colorimetric* (Anspec, Fremont, Calif.) The activity recovery was calculated based on Equation 1. The activity was fully recovered for cIAP after spray drying. The activity recovery value was higher than 100%, again possibly due to the variations caused by the non-optimized or non-validated enzymatic assay.

TABLE 6
Composition and enzymatic activity of spray dried cIAP

| | | |
|---|---|---|
| Percentage in | cIAP | 5.00% |
| formulation | HPMCAS HF | 87.45% |
| (w/w %) | Zinc sulfate heptahydrate | 0.06% |
| | Magnesium sulfate | 0.49% |
| | heptahydrate | |
| | Monosodium phosphate | 2.00% |
| | monohydrate | |
| | Trehalose | 5.00% |
| Spray drying | Inlet temperature | 145° C. |
| parameters | Outlet temperature | 46° C. |

| | |
|---|---|
| Activity recovery | 126% |

The sensitivity of cIAP to pH was investigated. It was found that cIAP was irreversibly and completely de-activated at gastric pH (pH 1-3), even in the presence of 1 mM magnesium ions and 0.1 mM zinc ions. At pH 4.0 and 5.0, cIAP lost about half of the activity without additional ions; but retained all the activity in the presence of both ions. Therefore, a good enteric protection is necessary to preserve IAP activity at extremely low pH and addition of magnesium and zinc ions to the formulation helps preservation of the IAP activity.

### REFERENCES

Fieker, A. Philpott, J., Armand, M. Enzyme replacement therapy for pancreatic insufficiency: present and future. Clin Exp Gastroenterol. 2011; 4: 55-73.

Jones, R. N., Wilson, H. W., Novick, Jr, W. J., Barry, A. L., Thornsberry, C. In vitro evaluation of CENTA, a new beta-lactamase-susceptible chromogenic cephalosporin reagent. J Clin Microbiol. 1982; 15(5): 954-958.

Leane, M. M., Sinclair, W., Qian, F., Haddadin, R., Brown, A., Tobyn, M., Dennis, A. B. Formulation and process design for a solid dosage form containing a spray-dried amorphous dispersion of ibipinabant. Pharmaceutical Development and Technology, 2012, 1-8, Early Online

Meng, F. T., Ma, G. H., Liu, Y. D., Qiu, W., Su, Z. G. Microencapsulation of bovine hemoglobin with high bio-activity and high entrapment efficiency using a W/O/W double emulsion technique. Colloids and Surfaces B: Biointerfaces 33 (2004) 177-183

Mudie D M, Amidon G L, Amidon G E. Physiological Parameters for Oral Delivery and In vitro Testing. Mol. Pharm., 2010; 7(5):1388-1405.

What is claimed is:

1. A process for formulating a protein or a peptide for oral delivery, the process comprising the steps of:
obtaining a powder containing the protein or peptide;
obtaining an amount of an enteric polymer, wherein the enteric polymer is neutralized so that it is soluble in water;
optionally obtaining at least one excipient;
mixing the protein or peptide, the neutralized enteric polymer, and optionally the at least one excipient to obtain a First Dispersion;
granulating the First Dispersion on a fluidized bed to obtain a Second Dispersion;
whereby the protein or peptide in the Second Dispersion undergoes less than 50% proteolysis under gastric conditions.

2. The process of claim 1, wherein the Second Dispersion is encapsulated in a capsule.

3. The process of claim 1, wherein the Second Dispersion is formulated as a powder for suspension or reconstitution, packaged as a sachet, or powder in a bottle.

4. The process of claim 1, wherein the binding polymer used to make the Second Dispersion is an enteric polymer.

5. The process of claim 4, wherein the enteric polymer is hydroxypropylmethylcellulose acetate succinate (HMPCAS), Grade H, M, or F.

6. The process of claim 1, wherein the binding polymer used to make the second Dispersion is a water soluble polymer.

7. The process of claim 1, wherein the binding polymer used to make the Second Dispersion is hydroxypropylmethyl Cellulose (HPMC).

8. The process of claim 1, wherein the First Dispersion powder is generated by being spray dried from a single aqueous solution.

9. The process of claim 1, wherein the Neutralized Enteric Polymer in the First Dispersion is the sodium salt of hydroxypropylmethylcellulose acetate succinate (HPMCAS), Grades H, M, or F.

10. The process of claim 1, wherein where the First Dispersion alone is used.

11. The process of claim 1, wherein the release of the API is modified by the addition of excipients.

12. The process of claim 1, wherein the added excipient is a solid acid.

13. The process of claim 1, wherein the added excipient is a solid base.
